(19) United States
(12) Patent Application Publication
(10) Pub. No.: US 2009/0299198 A1
(43) Pub. Date: Dec. 3, 2009

(54) ESTIMATING PULMONARY ARTERY DIASTOLIC PRESSURE

(75) Inventors: James K. Carney, Brooklyn Park, MN (US); Tommy D. Bennett, Shoreview, MN (US); Mustafa Karamanoglu, Fridley, MN (US)

Correspondence Address: MEDTRONIC, INC., 710 MEDTRONIC PARKWAY NE, MINNEAPOLIS, MN, 55432-9924, US

(73) Assignee: Medtronic, Inc.

(21) Appl. No.: 12/129,955

(22) Filed: May 30, 2008

(51) Int. Cl. A61B 5/0215; A61B 5/021

(52) U.S. Cl. 600/486; 600/485

(57) ABSTRACT

A method for estimating pulmonary artery diastolic pressure, for a single heart beat, includes establishing a time window for sampling and storing pressure data points from a right ventricular pressure transducer. The time window may be established according to predetermined parameters and/or according to one or more triggering events. An approximate time at which the pulmonary artery valve opens is determined, either via the sampled pressure data points, or via another form of more direct monitoring, during the time window, in order to estimate the pulmonary artery diastolic pressure. A plurality of sets of N pressure data points may be collected, from the sampled data, and, for each collected set, a weighted sum is calculated. Each weighted sum may be employed to evaluate a quality of the sampled data and/or to estimate the pulmonary artery diastolic pressure, if the more direct monitoring of the pulmonary artery valve is not employed.

### TECHNICAL FIELD

The present disclosure pertains to methods for ascertaining hemodynamic function, and more specifically to methods for estimating pulmonary artery diastolic pressure based, at least, upon pressures measured by a pressure transducer implanted in a right ventricle of a heart.

### BACKGROUND

A pressure transducer implanted in the right ventricle of a patient's heart may be used to measure right ventricular pressures for monitoring a heart condition, such as congestive heart failure, for example, to evaluate an efficacy of one or more therapies which are being administered to treat the heart condition. Those skilled in the art appreciate that pulmonary artery diastolic pressure (PAD) can be correlated to left ventricular filling pressure, and that an elevated PAD pressure can indicate a failure of the left ventricle to generate sufficient blood pressure to meet the needs of the patient, for example, due to congestive heart failure, pulmonary hypertension and/or mitral valve stenosis.

Right ventricular pressure data may be collected from the implanted pressure transducer and used to estimate PAD pressure; estimated PAD (ePAD) pressures, which are monitored over time, can facilitate the assessment of cardiac function for disease diagnosis and/or for therapy evaluation.

### BRIEF DESCRIPTION OF THE DRAWINGS

The following drawings are illustrative of particular embodiments of the disclosure and therefore do not limit the scope of the invention. The drawings are not to scale (unless so stated) and are intended for use in conjunction with the explanations in the following detailed description. Embodiments of the disclosure will hereinafter be described in conjunction with the appended drawings, wherein like numerals denote like elements.

FIG. 1A is a schematic depicting an exemplary implanted system, which includes a pressure transducer located in a right ventricle.

FIG. 1B is a block diagram for the system shown in FIG. 1A, according to some embodiments.

FIG. 2A-D are charts including plots of right ventricular and pulmonary artery pressures versus time.

FIG. 3 is a chart including representative plots of right ventricular pressure and pulmonary artery pressure, which are synchronized in time with a right ventricular electrocardiogram, and wherein alternative sampling time windows, according to some methods of the present invention, are shown.

FIG. 4A is a flow chart outlining some methods of the present disclosure.

FIG. 4B is a flow chart outlining some alternate methods of the present disclosure.

FIG. 5A is a flow chart outlining yet further methods of the present disclosure.

FIG. 5B is a schematic depicting collected pressure data points within a sampling time window, according to some methods.

### DETAILED DESCRIPTION

The following detailed description is exemplary in nature and is not intended to limit the scope, applicability, or configuration of the invention in any way. Rather, the following description provides practical illustrations for implementing exemplary embodiments. Utilizing the teaching provided herein, those skilled in the art will recognize that many of the examples have suitable alternatives that can be utilized.

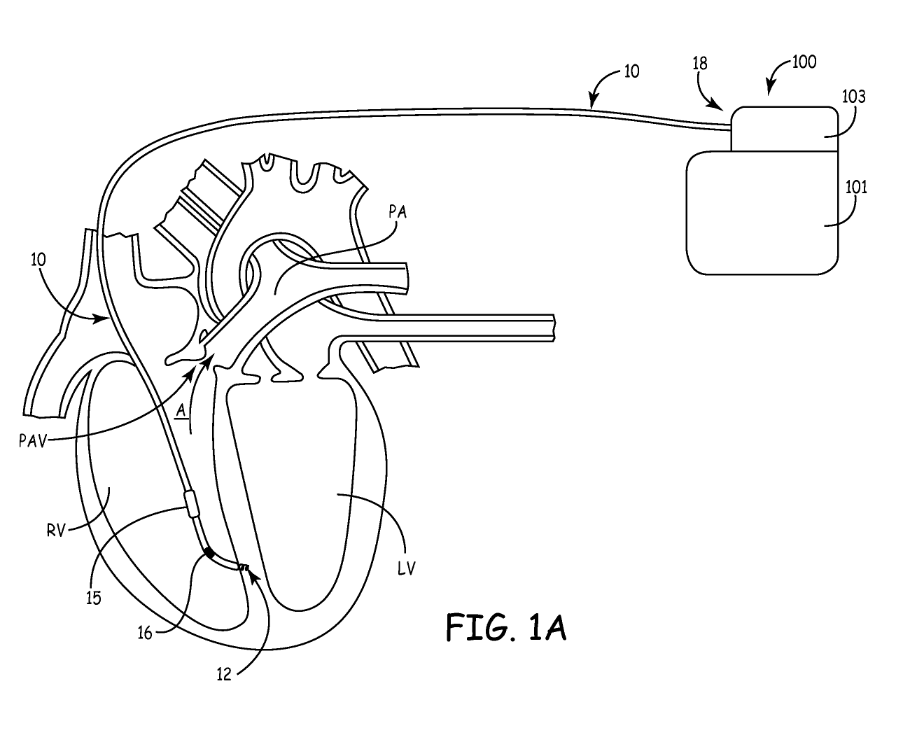

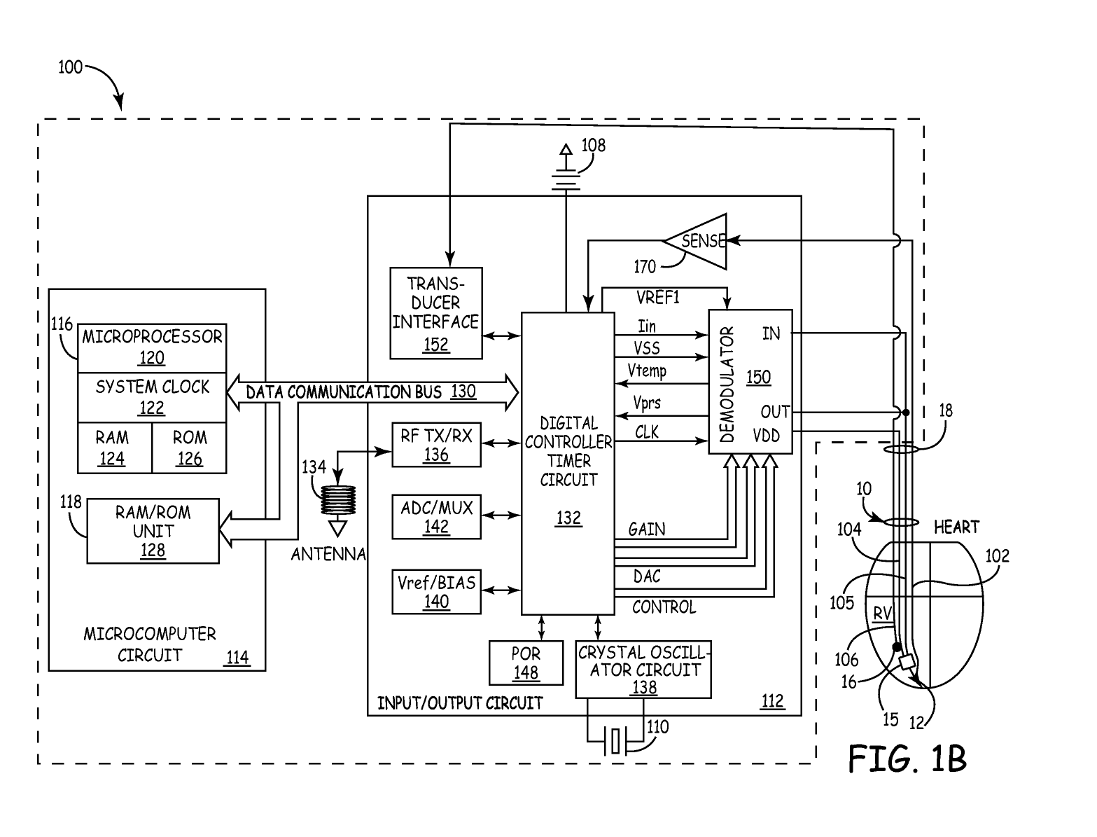

FIG. 1A is a schematic depicting an exemplary implanted system. FIG. 1A illustrates the implanted system including a device 100, for example, located in a pectoral region, and a medical electrical lead 10, which is connected to device 100, via a coupling between a proximal end 18 of lead 10 and a connector module 103 of device 100. FIG. 1A further illustrates lead 10 extending distally from device 100, within the venous system, to terminate at an implant site within a right ventricle RV of a heart; lead 10 is shown including a pressure transducer 15, an optional alternative transducer 16, and a fixation member 12, for example, a helix electrode, which is fastened to a septal wall of right ventricle RV in order to fix lead 10 at the implant site and to sense electrical activity of the heart muscle. Electrode 12 may further deliver pacing pulses to the heart muscle and, although not shown, lead 10 may include additional electrodes for example, to pace and/or sense, in conjunction with electrode 12, or to deliver alternative electrical stimulation. Those skilled in the art will appreciate that proximal end 18 of lead 10 includes connector contacts, which mate, for electrical coupling, with corresponding electrical contacts of device 100, which contacts are mounted within a bore of module 103. The electrical contacts of device 100 are in turn coupled to electronic circuitry contained within a hermetically sealed housing 101 of device 100, via hermetic feedthroughs, the construction of which are well known to those skilled in the art.

FIG. 1B is a block diagram for the system of FIG. 1A, according to some embodiments, wherein the containment of housing 101 is represented by dashed lines. FIG. 1B illustrates lead 10 including a pair of conductors 104, 105, which couple pressure transducer 15 to corresponding connector contacts at proximal end 18, another conductor 102, which couples electrode 12 to a corresponding connector contact at proximal end 108, and another conductor pair 106, which couple optional transducer 16 to corresponding connector contacts at proximal end 108. Those skilled in the art will appreciate that conductors 102, 104, 105, 106 may extend within a single lead, for example, lead 10, as illustrated in FIG. 1A, or may be partitioned among a plurality of leads. Pressure transducer 15 is shown coupled to a pressure signal demodulator 150 of an input/output circuit 112, via the coupling of the corresponding connector contacts with device contacts, electrode 12 is shown likewise coupled to a sense amplifier 170 of input/output circuit 112, and optional transducer 16 is shown likewise coupled to a corresponding transducer interface circuit 152 of input/output circuit 112. According to an exemplary embodiment of the present invention, pressure transducer 15 includes variable pickoff and fixed reference capacitors and signal modulating circuitry as is described in commonly-assigned U.S. Pat. No. 5,564,434, which is hereby incorporated by reference. Optional transducer 16 may be an accelerometer, an acoustic transducer, a Doppler flow transducer, or any other transducer known to those skilled in the art, which is suitable for triggering and/or monitoring functions, which functions may be employed, in conjunction with pressure sensing, according to some methods of the present invention. Input/output circuit 112 is further coupled to a battery 108, a crystal 110 and a telemetry antenna 134, and further includes a digital controller/timer circuit 132, a crystal oscillator 138, a power-on-reset (POR) circuit 148, a Vref/BIAS circuit 140, an ADC/MUX circuit 142 and an RF transmitter/receiver circuit 136; the function of each of these circuits is described in the aforementioned and incorporated-by-reference '434 patent, and is known to those skilled in the art. One function of digital controller/timer circuit 132 is to sample and digitize the pressure transducer signals from demodulator 150 using ADC/MUX circuit 142. The digital samples are then placed on a data communication bus 130. To those skilled in the art, it is known that the sampling and digitizing functions of demodulator circuit 150 and ADC/MUX circuit 142 can alternatively be carried out by circuitry that is incorporated into the structure of pressure sensor 15; in this case, the digitized pressure samples may be transmitted from sensor 15, via conductors 104, 105, to digital controller/timer circuit 132.

FIG. 1B further illustrates device 100 including a microcomputer circuit 114. Microcomputer circuit 114 is coupled to a set of timers and associated logic circuits of digital controller/timer circuit 132, via data communications bus 130, and includes an on-board circuit 116, with a microprocessor 120, a system clock 122, RAM 124 and ROM 126, and an off-board circuit 118 with a RAM/ROM unit 128 for additional memory capacity. Microprocessor 120 is preferably interrupt driven, operating in a reduced power consumption mode normally, and awakened in response to defined interrupt events, which may include a periodic timing out of data sampling intervals for storage of monitored data, the transfer of triggering and data signals on bus 130 and a receipt of programming signals. A real time clock and calendar function may also be included to correlate stored data to time and date. Microcomputer circuit 114 is further described in the aforementioned and incorporated-by-reference '434 patent.

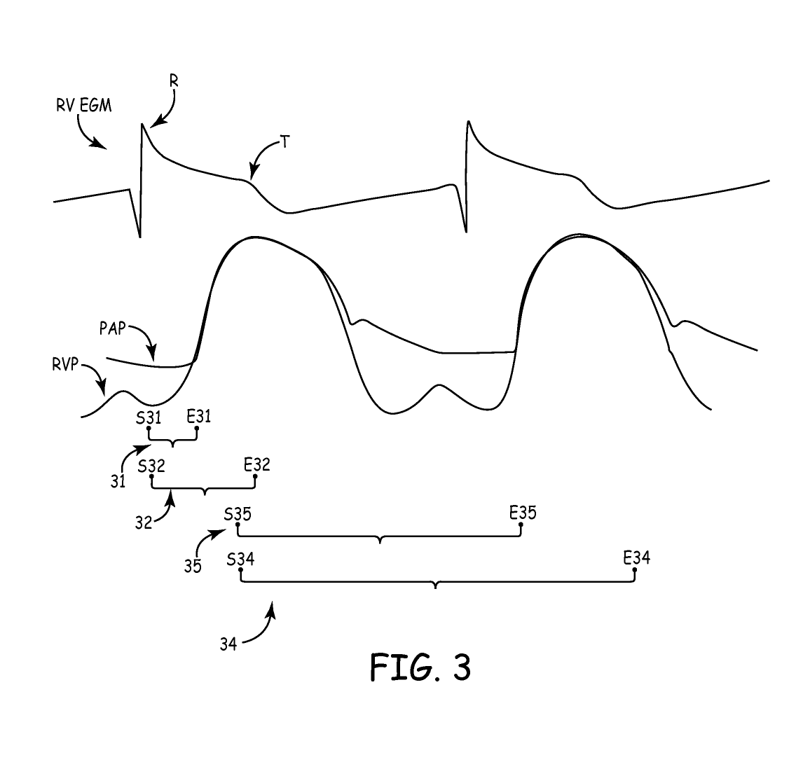

The system illustrated by FIGS. 1A-B may be employed to estimate pulmonary artery diastolic (PAD) pressure, according to various methods of the present invention, some of which will be described below in conjunction with FIGS. 3-6. However, it should be noted that alternative systems known in the art, for example those including one or more wireless sensors/transducers, may carry out methods of the present invention.

With reference back to FIG. 1A, it may be appreciated that a valve PAV of the pulmonary artery PA opens, in each cardiac cycle, to allow blood to be ejected from the right ventricle RV, along an outflow tract of the right ventricle RV, per arrow A; valve PAV opens at the time, in each cardiac cycle, when a pressure in the right ventricle RV just surpasses the pressure in the pulmonary artery PA. Thus, just prior to the opening of valve PAV, a pressure measured in the right ventricle RV, for example, by pressure transducer 15, is approximately equal to the pressure in the pulmonary artery PA when valve PAV opens, which pressure is approximately the lowest pressure within the pulmonary artery PA during the cardiac cycle and is called the pulmonary artery diastolic pressure, or PAD pressure, which was introduced above.

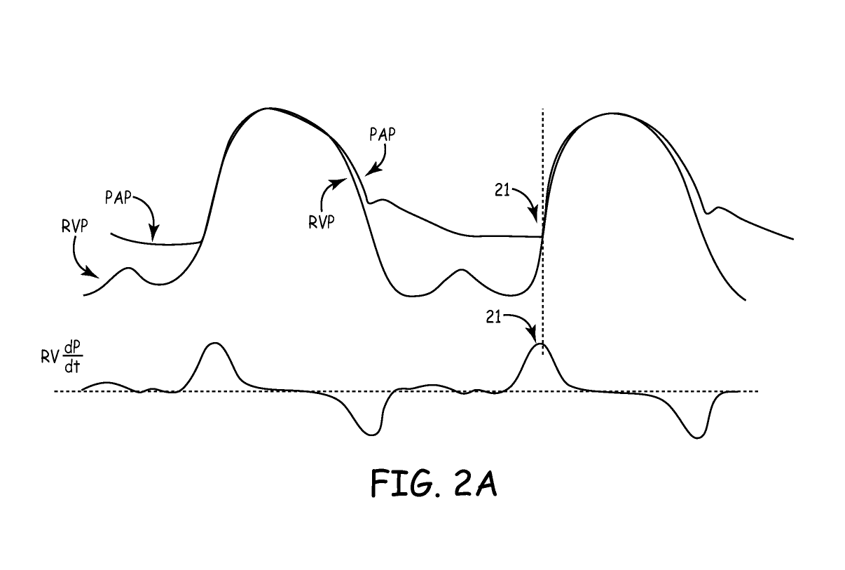

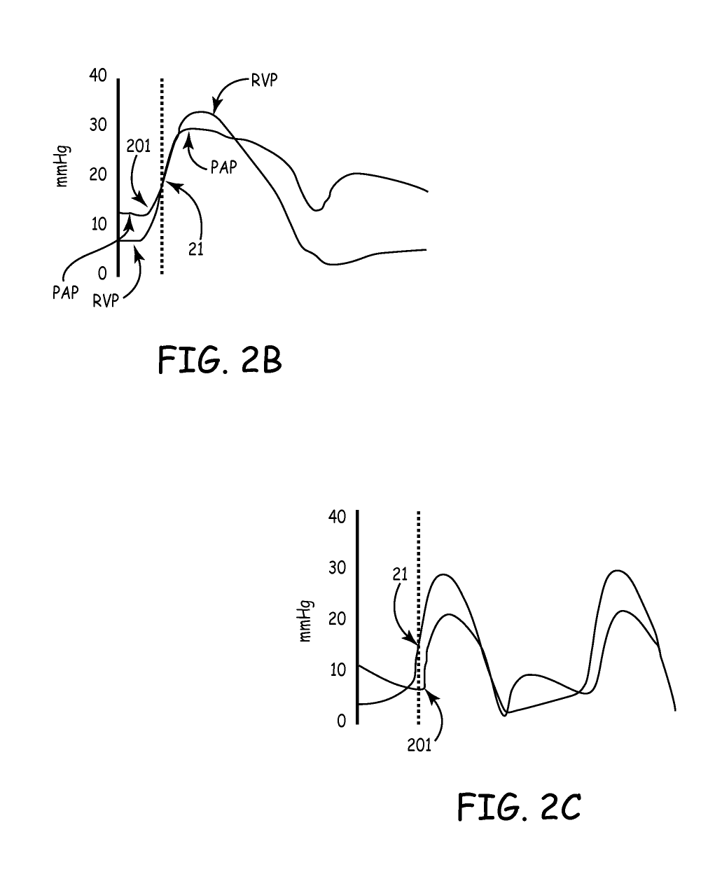

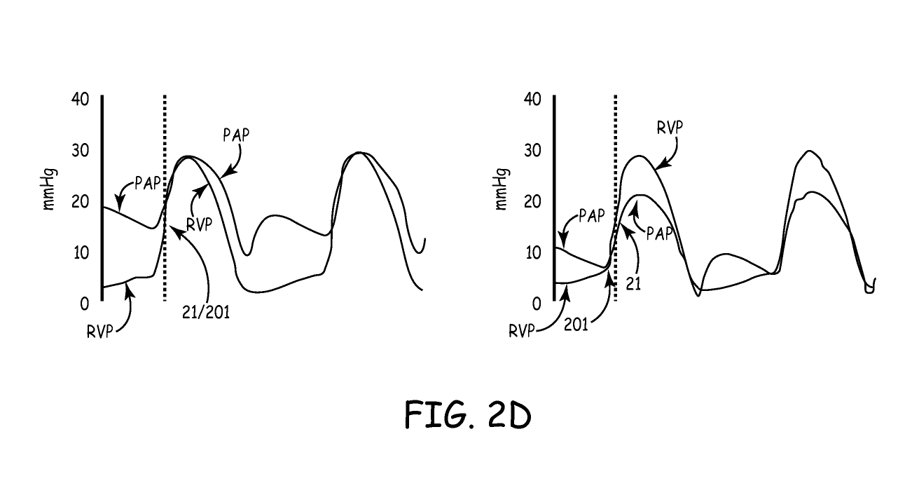

FIG. 2A is a chart, which includes plots of pulmonary artery pressure PAP and right ventricular pressure RVP, both versus time, which are representative of baseline, normal physiological function. FIG. 2A further includes another plot of the change in right ventricular pressure RVP with respect to time RVdP/dt, to illustrate how right ventricular pressure data points may be processed and monitored to estimate PAD pressure. A vertical dotted line, in FIG. 2A, marks a point in time 21 when the change in right ventricular pressure with respect to time RVdP/dt is maximum, which, according to the physiological conditions represented by the plots, coincides, in time, with the opening of valve PAV. With reference to FIG. 2A, it may be appreciated that a right ventricular pressure transducer may be employed by an implantable system, for example transducer 15 of the system illustrated in FIGS. 1A-B, to collect, store and process pressure data points in order to detect point in time 21, and then to designate the right ventricular pressure, which is measured at point in time 21, as the pulmonary diastolic (ePAD) pressure. However, under conditions other than baseline, normal physiological, the time of maximum RVdP/dt may not be as closely synchronized with the opening of valve PAV. Some examples of these conditions are illustrated in FIGS. 2B-2D.

FIG. 2B shows plots of right ventricular pressure RVP and pulmonary artery pressure PAP, wherein, due to increased myocardial contractility, point in time 21, at which the change in right ventricular pressure with respect to time RVdP/dt is maximum, lags a point in time 201 when valve PAV opens. The increased contractility causes pressure in the right ventricle RV to increase at a faster rate, and may be a result of inotropic therapy, for example, via administration of Dobutamine. FIG. 2C shows plots of right ventricular pressure RVP and pulmonary artery pressure PAP, wherein, due to a relatively high heart rate, point in time 21, at which the change in right ventricular pressure with respect to time RVdP/dt is maximum, precedes point in time 201 when valve PAV opens. FIG. 2D shows two sets of plots of right ventricular pressure RVP and pulmonary artery pressure PAP to show the impact that posture may have on the timing of the opening of valve PAV with respect to the time of maximum RVdP/dt. The plots on the left hand side of FIG. 2D are representative of a supine posture wherein points in time 21 and 201 approximately coincide, while the plots on the right hand side of FIG. 2D are representative of a standing posture wherein point in time 201 of valve PAV opening precedes point in time 21. Other factors which may cause a shift in the time of the opening of valve PAV from the time of maximum RVdP/dt include respiration and a relatively high right atrial pressure.

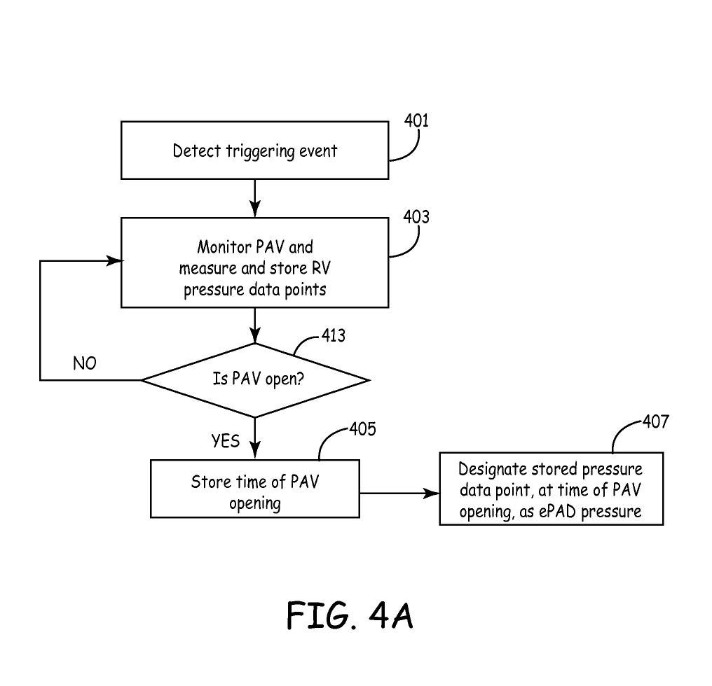

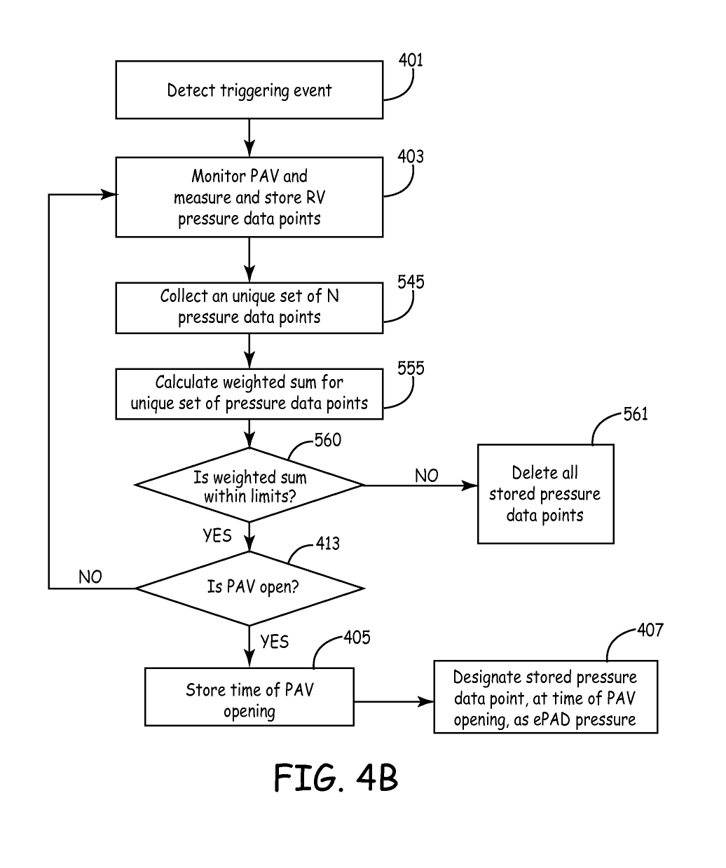

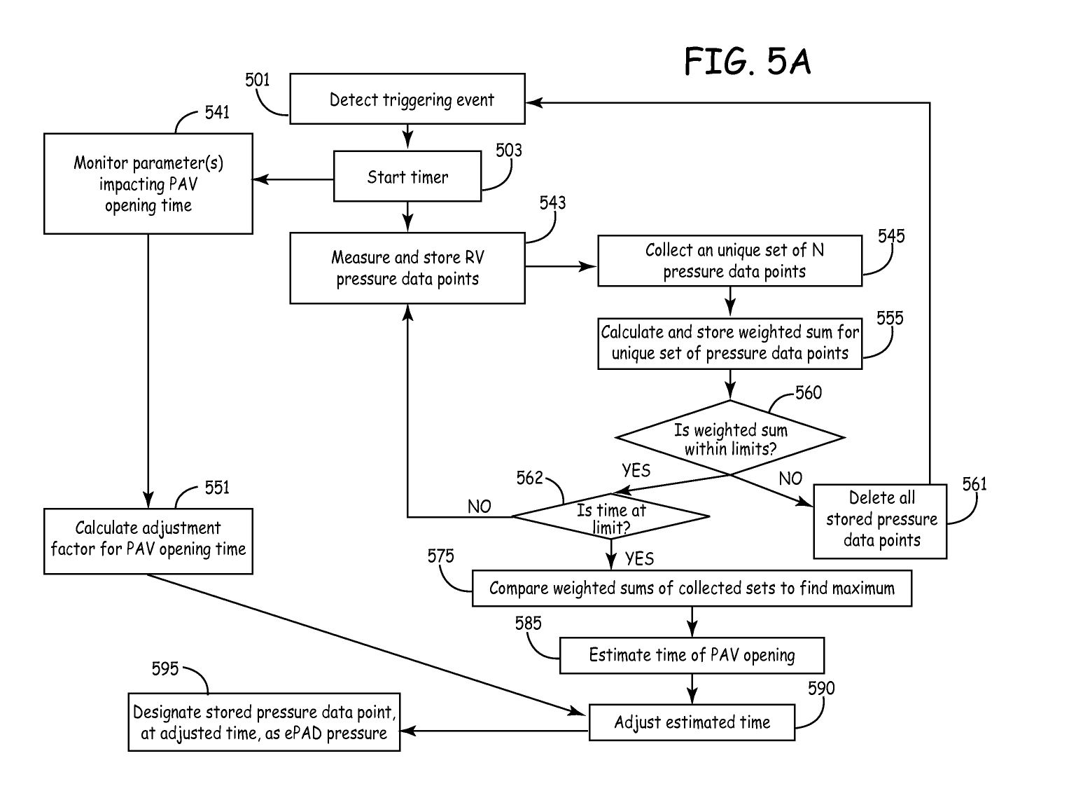

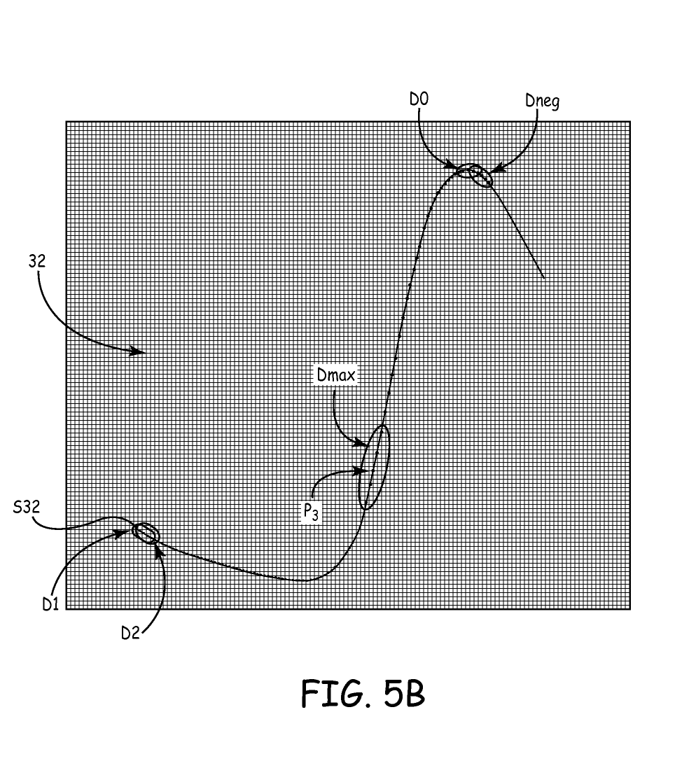

Although not encompassing all possible conditions, the plots of FIGS. 2B-D illustrate that a reliance on detection of the maximum change in right ventricular pressure with respect to time RVdP/dt alone, in order to estimate PAD pressure from right ventricular pressure RVP, could lead to significant errors in the estimate. Thus, some preferred methods of the present invention either incorporate an adjustment to the estimated time of the opening of valve PAV, when the estimated time is based on a detection of maximum RVdP/dt, or incorporate a more direct detection of the opening of valve PAV to estimate the time of valve opening. Flowcharts for the latter are illustrated in FIGS. 4 and 6, and a flowchart for the former, in FIG. 5A, all of which will be described below.

According to preferred methods of the present invention, rather than continuous signal monitoring and processing, signal sampling and processing steps are employed in discrete time windows, in order to reduce the consumption of energy, which is particularly desirable for downsized implantable systems that are designed for maximum longevity and minimum size. FIG. 3 is a chart including a representative plot of right ventricular pressure RVP and pulmonary artery pressure PAP, which are synchronized in time with an electrocardiogram EGM measured by an electrode, for example, electrode 12 of FIG. 1A, located in right ventricle RV, and wherein alternative sampling time windows 31, 32, 34 and 35 are shown. Electrical events of myocardial conduction, as sensed by electrode 12, of lead 10 (FIG. 1A), and represented by the EGM, may be used to establish at least starting points S31 and S32 of sampling time windows 31 and 32, respectively, according to some methods.

According to FIG. 3, respective starting points S31, S32 of sampling time windows 31, 32 may be triggered when electrode 12 senses ventricular depolarization, which is represented by an R-wave R on the EGM. Alternatively, if the system includes an electrode located for reliably sensing atrial depolarization, a detection of atrial depolarization may be used to trigger starting points S31, S32. According to other alternate embodiments, optional transducer 16 (FIGS. 1A-B) detects a mechanical event to trigger an alternative starting point for a sampling time window; examples of such mechanical triggering events include, without limitation, the first heart sound (vibration associated with the closure of mitral and tricuspid valves), for example, as measured by an acoustic transducer, and peak endocardial acceleration (during isovolumetric ventricular contraction), for example, as measured by an accelerometer.

An end point E31 of window 31 coincides with a detection of valve PAV opening, per some methods, as described below, in conjunction with FIGS. 4 and 6, while an end point E32 of sampling time window 32 may be a function of a predetermined duration for window 32, for example, as described below, in conjunction with FIG. 5A. According to additional alternate methods end point E32 may be triggered, like starting point S32, by an electrical event, or triggered by a mechanical event, which may be hemodynamic in nature, for example, as detected by pressure transducer 15 of lead 10, or related to heart sounds or heart wall motion, for example, as detected by optional transducer 16.

According to yet further alternate methods, sampling time windows may be opened at a predetermined time, for example, windows 34 and 35 of FIG. 3, which include starting points S34 and S35, respectively. Sampling time window 34 is shown including both a predetermined starting point S34 and a predetermined ending point E34, which are set apart from one another by a predetermined duration that is greater than the duration of one cardiac cycle, while sampling time window 35 is shown including an end point E35 triggered by detection of the opening of valve PAV, similar to end point E31 of window 31 described above. Sampling time window 34 may be pre-programmed to open up at a particular time of day for data sampling over a prescribed duration that encompasses at least one heart beat, in order to estimate PAD pressure.

It should be appreciated that sampling time windows 34, 35 may be programmed to open up for data sampling several times a day, or to open up less frequently. Likewise, sampling time windows 31, 32, even though including respective triggered starting points S31, S32, may be allowed to open several times in close succession, or less frequently, for example, once a day. It should be noted that windows 31, 32, 34 and 35 may span multiple heart beats, and that the methods which are described below, for a single heart beat, may be repeated over multiple successive heart beats, and then, resulting multiple estimates of PAD pressure averaged.

FIG. 4A is a flow chart outlining some methods of the present invention, wherein valve PAV is monitored, either directly, or by methods more direct than right ventricular pressure monitoring. FIG. 4A illustrates an initial step 401, wherein a triggering event is detected, for example, ventricular depolarization in the signal from electrode 12 detected by microprocessor 120 (FIG. 1B). Upon detection of the triggering event, a sampling window, such as window 31 (FIG. 3), is opened for pressure data sampling, for example, via activation of pressure signal demodulator 150, for processing the right ventricular pressure signal from transducer 15, and of microcomputer circuit 114, for pressure data point sampling (FIG. 1B), so that pressure data points may be measured and stored while valve PAV is monitored, per step 403. According to some methods, a delay may be pre-programmed between steps 401 and 403. Alternatively, step 403 is begun at a predetermined time, for example, according to sampling time window 35 (FIG. 3), as previously described, so that step 401 is not necessary.

Valve PAV may be directly monitored, per step 403, for example, via ultrasound imaging; alternatively methods may be employed, within step 403, to monitor parameters, which change as a result of the opening of valve PAV. Some of these parameters, which may be monitored in order to detect the opening of valve PAV, are those indicative of blood flow out from the right ventricle RV; these parameters include, without limitation: flow itself, for example, measured by a flow probe located in proximity to the outflow tract of the right ventricle RV; local temperature, for example, measured by pressure transducer 15 according to an embodiment described in the aforementioned and incorporated-by-reference U.S. Pat. No. 5,564,435; and impedance, for example, measured between two electrodes located in the right ventricle RV. While valve PAV is being monitored, right ventricular pressure data points are sampled and stored until the opening of valve PAV is detected, per decision step 413, at which time pressure data sampling is ended and the time of detected valve PAV opening is stored, per step 405. The stored pressure data point, which coincides in time with the detection of the opening, is then designated as ePAD pressure, per step 407.

According to alternate methods, the pressure data points, which are stored in step 403, are also processed, either in real time, or just subsequent to detection of the opening of valve PAV, in order to assure that the stored pressure data points are not distortions caused by artifacts on the pressure transducer signal. If the pressure data points are suspect, the stored data may be dumped and then the steps of FIG. 4A repeated. FIG. 4B is a flow chart outlining such an alternative method.

FIG. 4B illustrates data processing beginning at a step 545, wherein the sampled data is collected into unique sets of N pressure data points, and then, per step 555, a weighted sum of the N points of each unique data set is calculated and stored. Each unique set may, or may not, overlap another of the sets. According to some methods, the weighting factors for the first and last points of each unique set are set are equal to negative 1 and positive 1, respectively, while the weighting factors for all of the intermediate points of each set are set equal to zero. Thus, it may be appreciated that a change in right ventricular pressure with respect to time RVdP/dt, for each set, is estimated by the corresponding weighted sum, which is essentially the difference in magnitude between the Nth pressure data point and the first pressure data point. In order to account for noise on the pressure signal, from which the data points are sampled, according to some alternate methods, some of the intermediate points are weighted for inclusion in the weighted sum, for example, the second point weighted at negative 0.5 and the N−1 point weighted at positive 0.5. At decision step 560, each weighted sum is checked against an upper limit and a lower limit, which are established, according to a knowledge of physiological right ventricular pressure changes; the upper limit can filter out mechanical artifacts, which may cause spikes in the pressure signal, and the lower limit can filter out pre-ventricular contractions.

According to FIG. 4B, the time at which the opening of valve PAV is detected is not stored unless each calculated weighted sum, per step 555, is found to be within the predetermined limits, per decision step 560. If no distortion of the pressure data is detected, then that stored pressure data point, which coincides in time with the stored time of valve PAV opening is designated as ePAD pressure, per step 407. If any of the weighted sums is not within the limits, all of the pressure data, which has been stored, is deleted, per a step 561, and step 403 is restarted, upon detection of a subsequent triggering event, per step 401.

FIG. 5A is a flow chart outlining yet further methods of the present invention, wherein valve PAV is more indirectly monitored, via the change in right ventricular pressure with respect to time RVdP/dt; and FIG. 5B is a schematic depicting sampled pressure data points within a sampling time window, such as window 32 of FIG. 3, which may be processed to determine the time of maximum RVdP/dt, via the data processing steps 545, 555 and 560, previously described, in combination with steps 575 and 585 of FIG. 5A. FIG. 5A illustrates detection of a triggering event, per a step 501, which directs a timer to start, per a step 503. According to some methods, a delay may be pre-programmed between steps 501 and 503. Alternatively, data sampling and monitoring is begun at a predetermined time, for example, according to sampling time window 34 (FIG. 3), as previously described, so that step 501 is not necessary. Once the timer is started, per step 503, pressure data sampling begins and right ventricular pressure data points are measured and stored, per a step 543, and processed, starting at step 545. The pressure data sampling rate may be approximately 256 samples per second, and the timer limit may be set, for example, to approximately 400 milliseconds; the predetermined duration is preferably established according to knowledge of the duration of each cardiac cycle for an expected average heart rate of the patient in which the system is implanted.

According to FIG. 5A, an initial stage of data processing, which includes steps 545, 555, 560 and 562, takes place while the timer runs and while pressure data points are being sampled and stored, per step 543; this first stage of data processing may be terminated either at decision step 560, or when the timer is at its limit, per decision step 562. Alternatively, this first stage of data processing may take place after the timer has reached its limit and the data sampling window is closed. FIG. 5A further illustrates a step 541, in which one or more parameters, impacting the time at which valve PAV opens, are monitored; step 541 takes place in parallel with steps 543, 545, 555, 560 and 562, so that an adjustment factor for valve PAV opening time may be calculated, per a step 551, for a latter stage of data processing. Although preferred methods include steps 541 and 551, alternate embodiments need not include these steps.

In step 545, the sampled data is collected into unique sets of N pressure data points (N=5, according to FIG. 5B), and then, per step 555, the weighted sum of the N points of each unique data set is calculated and stored, as previously described in conjunction with FIG. 4B. With reference to FIG. 5B a pair of exemplary data sets D1 and D2 are shown, to illustrate a preferred method of grouping the pressure data points, wherein each subsequent set overlaps the previous set by N−1 pressure data points. However, it should be noted that according to alternate methods, the subsequent sets may overlap by a fewer number of data points, or may not overlap at all. At decision step 560, each weighted sum is checked against an upper limit and a lower limit, as previously described, and, if the weighted sum is not within the limits, all of the pressure data, which has been stored, since the start of the timer, is deleted, per a step 561, and pressure data point collection and processing are restarted, as well as the monitoring of step 541, upon detection of a subsequent triggering event, per step 501. If the weighted sum is within the limits, then the timer is checked, per decision step 562, and, if the timer has not reached its limit, the storage of pressure data points, per step 543, the processing of pressure data points, starting at step 545, and the monitoring, per step 541, continue. Thus, a plurality of unique sets of N pressure data points are collected and evaluated by repeating steps 545, 555 and 560 until the timer is at its limit. As previously mentioned, the processing of pressure data points, per steps 545, 555 and 560, may be accomplished by grouping sets of points collected from storage, after the timer reaches its limit, as opposed to in parallel with data storage per step 543.

Once all the weighted sums, one for each of the plurality of sets, have been calculated, they are compared with one another to find a maximum, per step 575, and then a time of the opening of valve PAV, per a step 585, may be estimated. The estimated time of the opening of valve PAV may correspond to a midpoint of the set having the maximum weighted sum, for example, a point P3 of a data set Dmax, which is shown in FIG. 5B. According to some methods, this pressure may be designated as ePAD pressure. But, for a number of reasons, examples of which were previously described in conjunction with FIGS. 2B-D, the estimated time of the opening of valve PAV, based upon this maximum weighted sum, which generally corresponds to the maximum change in right ventricular pressure with respect to time RVdP/dt, may be in significant error such that the right ventricular pressure, which was measured at this estimated time, is not a good estimate of PAD pressure. So, according to preferred methods, outlined in FIG. 5B, an adjustment is made to the estimated time, per a step 590, according to the adjustment factor calculated in a step 551. The adjustment factor is calculated, per step 551, according to the one or more parameters, which are monitored in step 541, during pressure data point storage and the initial stage of pressure data point processing. The one or more monitored parameters may include heart rate, atrial pressure, respiration, posture and the rate of change in right ventricular pressure RVdP/dt itself, which were previously described, in conjunction with FIGS. 2B-D, as impacting the time of valve PAV opening with respect to the time at which RVdP/dt is at its maximum. Once the estimated time is corrected, or adjusted, per step 590, a stored pressure data point, which coincides with, or approximately coincides with, the adjusted time, or a pressure value between two stored pressure data points that each approximately coincide with the adjusted time, may be selected as ePAD pressure, per a step 595.

It should be reiterated, that, according to alternate embodiments, a duration of a sampling time window may not be predetermined, so that step 503 and decision step 562 of the method outlined in FIG. 5A would be replaced by a step to monitor another triggering event and decision step to end the initial stage of data processing (steps 545, 555 and 560), upon detection of the other triggering event. The other triggering event may be electrical or mechanical. An electrical triggering event may be the detection of cardiac repolarization, represented by a T-wave T on the EGM (FIG. 3). One mechanical triggering event may be the detection of peak right ventricular pressure, either by a calculated change in right ventricular pressure being close to zero, for example, shown for a data set D0 of FIG. 5B, or by a calculated negative change in right ventricular pressure, for example, shown for data set Dneg of FIG. 5B. Another mechanical triggering event may be the detection of the second heart sound (vibration associated with the closure of aortic and pulmonary valves).

As previously mentioned, successive estimates of PAD pressure, by any of the methods described herein, over a relatively short period of time may be desirable in order to increase an accuracy of the estimates, by calculating an average of the successive estimates. Furthermore, PAD pressure may be repeatedly estimated, according to any of the methods described herein, over a period of days to months, in order to monitor a heart condition, for example, congestive heart failure, and/or an efficacy of one or more therapies which are being administered to treat the heart condition.

In the foregoing detailed description, the invention has been described with reference to specific embodiments. However, it may be appreciated that various modifications and changes can be made without departing from the scope of the invention as set forth in the appended claims.

1. A method for estimating pulmonary artery diastolic pressure for a single heart beat, the method comprising:
establishing a time window for sampling pressure data points of a pressure signal provided by a pressure transducer implanted in a right ventricle of a heart;
sampling the pressure data points;
storing the sampled pressure data points;
collecting a plurality of unique sets of N pressure data points from the sampled pressure data points;
calculating a weighted sum for each collected set of N pressure data points, each weighted sum being indicative of a change in pressure across the corresponding collected set;
storing the weighted sum for each set;
comparing the weighted sums with one another to find a maximum weighted sum; and
selecting a pressure value from within a range of the pressure data points in the set having the maximum weighted sum to estimate pulmonary artery pressure.

2. The method of claim 1, wherein establishing the time window for sampling the pressure data points comprises predetermining the duration of the time window.

3. The method of claim 2, wherein the predetermined duration of the time window is greater than a duration of one heart beat.

4. The method of claim 2, wherein establishing the time window for sampling the pressure data points further comprises starting the time window upon detection of an event.

5. The method of claim 1, wherein establishing the time window for sampling the pressure data points comprises starting the time window upon detection of an event.

6. The method of claim 5, wherein the event comprises an electrical myocardial conduction event.

7. The method of claim 5, wherein the event comprises a mechanical event.

8. The method of claim 1, wherein establishing the time window for sampling the pressure data points comprises terminating the time window upon detection of an event.

9. The method of claim 8, wherein the event comprises an electrical myocardial conduction event.

10. The method of claim 8, wherein the event comprises a mechanical event.

11. The method of claim 1, wherein establishing the time window for sampling the pressure data points comprises predetermining a start time for the window.

12. The method of claim 1, wherein the selected pressure data point is an approximate midpoint of the set having the maximum weighted sum.

13. The method of claim 1, further comprising comparing each of the weighted sums to upper and lower limits, and, if any of the weighted sums is outside the upper and lower limits, stopping the sampling and deleting the stored sampled data points.

14. The method of claim 1, further comprising:
storing a time corresponding to the selected pressure data value, the time being an estimated time at which the pulmonary artery valve opens;
adjusting the stored time by a factor related to a monitored parameter, the monitored parameter being one that impacts a time at which the pulmonary artery valve opens; and
selecting another pressure value from within a range of the stored pressure data points, that corresponds to the adjusted time, to estimate the pulmonary artery pressure.

15. A method for estimating pulmonary artery diastolic pressure for a single heart beat, the method comprising:
establishing a time window for sampling pressure data points of a pressure signal provided by a pressure transducer implanted in a right ventricle of a heart;
sampling the pressure data points;
storing the sampled pressure data points;
collecting at least one set of N pressure data points from the sampled pressure data points;
calculating a weighted sum for each collected set of N pressure data points, each weighted sum being indicative of a change in pressure across the corresponding collected set;
comparing each weighted sum to a predetermined upper and lower limit, and, if any weighted sum is outside the upper and lower limits, deleting all the stored sampled pressure data points;
monitoring the pulmonary artery valve while sampling the pressure data points, until either an opening of the valve is detected or until all the stored sampled pressure points are deleted;
storing the time of detected opening of the valve, if the stored sampled pressure points have not been deleted; and
estimating the pulmonary artery pressure to be a value from within a range of the stored pressure data points that corresponds in time to the stored time of detected opening.

16. The method of claim 15, wherein establishing the time window for sampling pressure data points comprises starting the time window upon detection of an event.

17. The method of claim 16, wherein the event comprises an electrical myocardial conduction event.

18. The method of claim 16, wherein the event comprises a mechanical event.

19. The method of claim 15, wherein establishing the time window for sampling pressure data points comprises pre-determining a start time for the window.

20. The method of claim 15, wherein monitoring the pulmonary artery valve comprises direct monitoring via ultrasound imaging.

21. The method of claim 15, wherein monitoring the pulmonary artery valve comprises monitoring a parameter indicative of blood flow out from the right ventricle.

22. A method for monitoring a heart condition, comprising:
estimating a pulmonary artery diastolic pressure, for a single heart beat, at successive points in time according to the following steps, which are repeated at predetermined intervals:
establishing a time window for sampling pressure data points of a pressure signal provided by a pressure transducer implanted in a right ventricle of a heart;
sampling the pressure data points;
storing the sampled pressure data points;
collecting a plurality of unique sets of N pressure data points from the sampled pressure data points;
calculating a weighted sum for each collected set of N pressure data points, each weighted sum being indicative of a change in pressure across the corresponding collected set;
storing the weighted sum for each for each set;
comparing the weighted sums with one another to find a maximum weighted sum; and
selecting a pressure value from within a range of the pressure data points in the set having the maximum weighted sum to estimate pulmonary artery pressure.

23. The method of claim 22 wherein the steps for estimating the pulmonary artery diastolic pressure further comprise:
storing a time corresponding to the selected pressure value, the time being an estimated time at which the pulmonary artery valve opens;
adjusting the stored time by a factor related to a monitored parameter, the monitored parameter being one that impacts a time at which the pulmonary artery valve opens; and
selecting another pressure value from within a range of the stored pressure data points, that corresponds to the adjusted time, to estimate the pulmonary artery pressure.

24. An implantable medical device (IMD) configured to estimate pulmonary artery diastolic pressure for a heart beat, the IMD comprising;
means for establishing a time window for sampling pressure data points of a pressure signal provided by a pressure transducer implanted in a right ventricle of a heart;
means for sampling the pressure data points;
means for storing the sampled pressure data points;
means for collecting a plurality of unique sets of N pressure data points from the sampled pressure data points;
means for calculating a weighted sum for each collected set of N pressure data points, each weighted sum being indicative of a change in pressure across the corresponding collected set;
means for storing the weighted sum for each set;
means for comparing the weighted sums with one another to find a maximum weighted sum; and
means for selecting a pressure value from within a range of the pressure data points in the set having the maximum weighted sum to estimate pulmonary artery pressure.
